(19) United States
(12) Patent Application Publication
(10) Pub. No.: US 2010/0227384 A1
(43) Pub. Date: Sep. 9, 2010

(54) Portable Genomic Analyzer

(75) Inventor: Charles S. VANN, Marietta, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/698,093

(22) Filed: Feb. 1, 2010

Related U.S. Application Data
(63) Continuation of application No. 11/453,340, filed on Jun. 14, 2006, now abandoned.
(60) Provisional application No. 60/704,891, filed on Aug. 2, 2005.

(51) Int. Cl. C12M 1/34

(52) U.S. Cl. 435/287.2

(57) ABSTRACT

An apparatus for identifying a target portion of a sample. In some embodiments, the sample can be substantially purified and/or amplified in a test chamber. In some embodiments, the sample can be provided in an amount so that amplification is not necessary. The apparatus generally comprises a test chamber having an input region for receiving a sample and an analysis region. A sample chamber can be defined by at least a portion of the test chamber. A separation mechanism can separate a target portion of the sample from the sample and an analysis chamber can aid in analyzing at least the target portion of the sample.

### CROSS-REFERENCE TO RELATED APPLICATIONS

This application claims the benefit of U.S. Provisional Application No. 60/704,891, filed on Aug. 2, 2005. The disclosure of the above application is incorporated herein by reference.

### INTRODUCTION

Currently, genomic analysis, including that of the estimated 30,000 human genes, is a major focus of basic and applied biochemical and pharmaceutical research. Such analysis may aid in developing diagnostics, medicines, and therapies for a wide variety of disorders. However, the complexity of the human genome and the interrelated functions of genes often make this task difficult. There is a continuing need for methods and apparatus to aid in such analysis.

### BRIEF DESCRIPTION OF THE DRAWINGS

The skilled artisan will understand that the drawings, described herein, are for illustration purposes only. The drawings are not intended to limit the scope of the present teachings in any way.

FIG. 1 is a perspective view illustrating a genomic analyzer according to some embodiments of the present teachings;

FIG. 2 is a schematic plan view of a test chamber in accordance with some embodiments;

FIG. 3 is a schematic view of a filled sample chamber in the test chamber in accordance with some embodiments;

FIG. 4 is a schematic view of a sample moved from a sample chamber to a lysing chamber and lysed in accordance with some embodiments;

FIG. 5 is a schematic view of a portion of a sample moved from a lysing chamber to a PCR chamber and amplified therein in some embodiments;

FIG. 6A is a schematic view of an amplicon moving to a ligation chamber in some embodiments;

FIG. 6B is a schematic view of a ligation processor that may occur in the ligation chamber in some embodiments;

FIG. 7 is a schematic view of beads being centrifuged towards an analysis portion of the test chamber in some embodiments;

FIG. 8 is a schematic view of an analysis configuration in some embodiments;

FIG. 9 is an exemplary output image of the system; and

FIG. 10 is a schematic view of a test chamber in some embodiments.

### DESCRIPTION OF SOME EMBODIMENTS

The following description of some embodiments is merely exemplary in nature and is in no way intended to limit the present teachings, applications, or uses. Although the present teachings will be discussed in some embodiments as relating to polynucleotide amplification, such as PCR, such discussion should not be regarded as limiting the present teaching to only such applications.

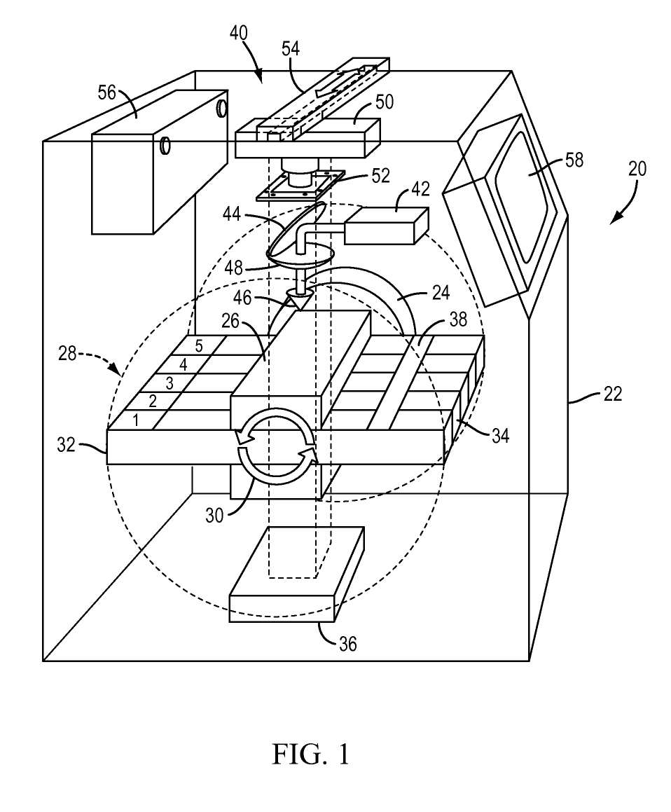

With reference to FIG. 1, a genomic analyzer or detector 20 is illustrated in accordance with some embodiments of the present teachings. It should be understood, however, that genomic analyzer 20 can be designed, assembled, constructed, and/or otherwise provided in any appropriate package or configuration.

In some embodiments, genomic analyzer 20 can comprise a housing or envelope system 22, a motor 24, an axle or chamber grasping system 26, a test chamber 28, and a mixer 36, or any combination thereof. In some embodiments, housing 22 can enclose or otherwise contain these portions of genomic analyzer 20 and/or can be sized to permit portability by a single user.

Still referring to FIG. 1, in some embodiments, motor 24 can be operably interconnected with axle or chamber grasping system 26. In this regard, motor 24 can operably drive chamber grasping system 26 to rotate or move test chamber 28. In some embodiments, motor 24 operably moves test chamber 28 in a predetermined manner, such as in a rotational direction indicated by arrow 30, about an axis defined along chamber grasping system 26. In this regard, an input end 32 of test chamber 28 generally travels around a circle defined by test chamber 28. Similarly, an output or analysis end 34 of test chamber 28 generally travels through the circle defined by test chamber 28. However, in some embodiments, a drive mechanism can be employed to cause test chamber 28 to travel along any one of a number of paths, such as but not limited to, an elliptical path.

In some embodiments, mixer 36 can be employed to mix a sample disposed within test chamber 28 by rotating, via motor 24, test chamber 28 to a position generally adjacent mixer 36. In this regard, mixer 36, such as an ultrasonic mixer, can be activated to mix the contents of test chamber 28, such as through the use of ultrasonic sound waves, which agitate or otherwise mix the contents of test chamber 28.

In some embodiments, analyses and/or various procedures can be performed within test chamber 28 before, during, and/or after testing. For example, in some embodiments, a thermocycler 38 can be provided to change and/or cycle a temperature of at least a portion of test chamber 28. It should be understood that thermocycler 38 can be positioned in any appropriate position relative to test chamber 28 to achieve at least partial thermal contact. In some embodiments, thermocycler 38 can be used in performing selected analysis and/or procedures, such as Polymerase Chain Reaction (PCR). In some embodiments, thermocycler 38 can be a resistive strip extending along a portion of test chamber 28 to impact thermal communication therewith.

Furthermore, in some embodiments, once one or more analyses and/or procedures have occurred, the results or output of these procedures can be determined and/or analyzed using an analysis assembly 40. Analysis assembly 40, in some embodiments, can comprise a laser 42 operable to illuminate and/or irradiate at least a portion of test chamber 28. For example, laser 42 can be used to direct a laser beam 46 at a selected wavelength, which can comprise a color of visible light in some embodiments, at analysis end 34 of test chamber 28. One or more dichroic mirrors 44 can be provided to assist in directing laser beam 46 towards analysis end 34 of test chamber 28. In some embodiments, laser 42 can be replaced with an alternative excitation source, such as an Argon ion laser, an LED, a halogen bulb, or any other known source.

In some embodiments, genomic analyzer 20 can further comprise various other optical members, such as a lens 48. In some embodiments, lens 48 can focus laser beam 46 from laser 42 upon test chamber 28, as well as any emission that emanates from test chamber 28.

In some embodiments, analysis assembly 40 can comprise a camera 50 operable to detect and/or gather fluorescence or other emanating energy from test chamber 28 and/or dichroic mirror 44. In some embodiments, a selection mechanism, such as a filter 52, can be used to assist in selecting a desired wavelength of energy to reach camera 50. In other words, filter 52 permits a desired wavelength (i.e. color) of energy to pass therethrough. As described herein, the wavelength of energy detected and/or gathered by camera 50 can be used to determine the presence of a selected target. In some embodiments, camera 50 can be coupled with a motor 54 to permit camera 50 to be moved relative to analysis end 34 of test chamber 28. In some embodiments, motor 54 can move camera 50 among or between two or more test chambers 28.

In some embodiments, laser 42 can output laser beam 46 at a wavelength sufficient to irradiate and/or excite a selected analytical substance, such as one or more probes, that in turn radiates, fluoresces, or otherwise outputs energy at a known wavelength for detection.

In some embodiments, genomic analyzer 20 comprises a power source 56. In some embodiments, power source 56 can be self-contained, such as a battery. Such self-contained power source 56 can permit a user to move genomic analyzer 20 closer to a source of a sample for ease of use. In some embodiments, genomic analyzer 20 can comprise a power converter that can be connected to an external power source. In any case, power source 56 can be provided to permit simple and convenient portability of genomic analyzer 20.

In some embodiments, genomic analyzer 20 comprises a user panel 58 that can output results of the analysis performed with genomic analyzer 20 for review by a user and/or query the user regarding further action. In some embodiments, user panel 58 of genomic analyzer 20 can comprise an input device. In some embodiments, this input device can be separate from genomic analyzer 20. The input device can permit a user to program genomic analyzer 20 for a selected procedure. For example, the user can program genomic analyzer 20 to perform a certain number of thermocycles. Therefore, in some embodiments, user panel 58 can both display a result and permit the user to program genomic analyzer 20. In some embodiments, genomic analyzer 20 can comprise an output device that permits output from genomic analyzer 20 to be later processed and/or displayed in a human readable output, such as a printer or monitor.

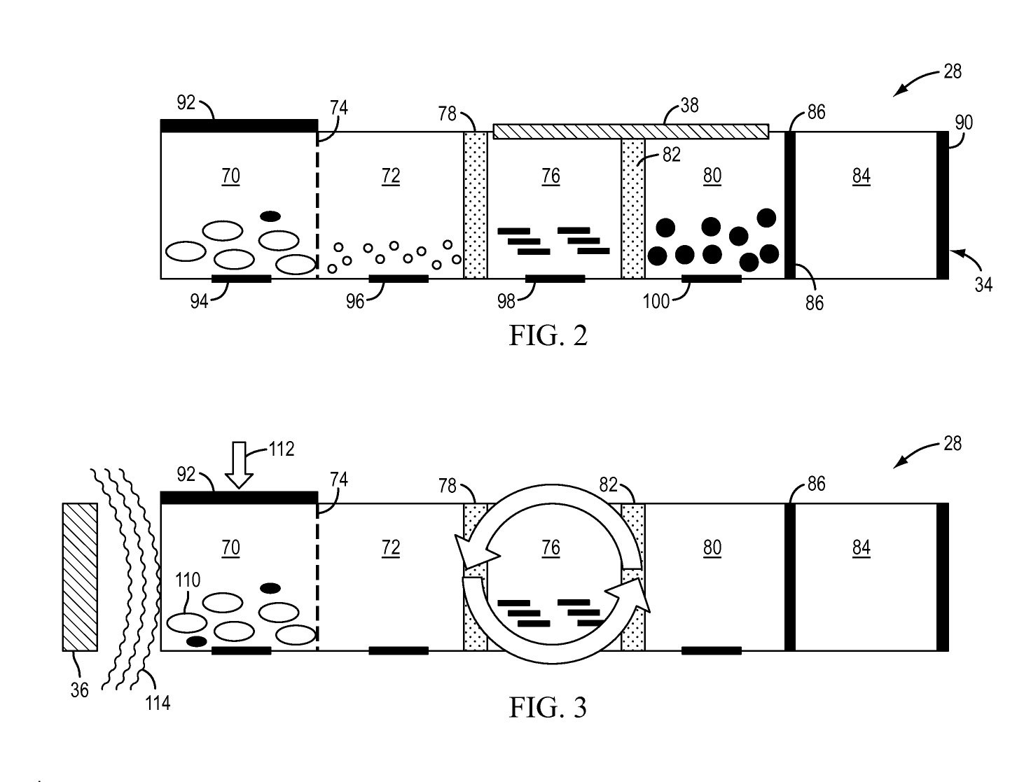

Referring to FIG. 2, in some embodiments, test chamber 28 comprises a plurality of sub-chambers, such as a sample chamber 70, a lysing chamber 72, a PCR chamber 76, a ligation chamber 80, a detection chamber 84, or any combination thereof. In some embodiments, the plurality of sub-chambers 70, 72, 76, 80, and 84 can be separated to permit portions of the preparation and/or analysis of sample 110 to be performed discretely and sequentially. Sample chamber 70 can be sized to receive a volume of sample 110 for processing and/or analysis. In some embodiments, lysing chamber 72 can be provided to lyse at least a portion of sample 110. In some embodiments, a first filter or membrane 74 can separate sample chamber 70 and lysing chamber 72 to permit an initial or first separation of a target element or target sample 110₊ from sample 110. For example, in some embodiments, first filter 74 can comprise a selected pore size such that only a selected size of material is able to pass through first filter 74 from sample chamber 70 into lysing chamber 72.

In some embodiments, amplification or polymerase chain reaction (PCR) chamber 76 can be provided across a second filter 78 from lysing chamber 72. That is, in some embodiments, second filter 78 can comprise a gel matrix that can permit a selected portion of sample 110 to pass into PCR chamber 76. It should be understood that PCR chamber 76 can be provided to perform any appropriate amplification of a selected portion of sample 110, such as a DNA portion, RNA portion, or combinations thereof. In some embodiments, ligation chamber 80 can be provided across a third filter 82 that can also include a gel matrix. Third filter 82 can be used to permit a selected portion of sample 110 to pass from PCR chamber 76 to ligation chamber 80.

In some embodiments, thermocycler 38 can be provided adjacent PCR chamber 76 and ligation chamber 80 to thermally cycle sample 110. In some embodiments, thermocycler 38 can be a resistive strip adhered or fixed to test chamber 28. In some embodiments, thermocycler 38 can comprise a device positioned in housing 22 to cycle a temperature therein and in test chamber 28.

In some embodiments, detection chamber 84 can be provided near analysis end 34 of test chamber 28. Detection chamber 84 can comprise portions for use with analysis assembly 40 for a detection of sample 110 or target sample 110₊ of sample 110. In some embodiments, a movable or breakable member 86 can physically separate ligation chamber 80 from detection chamber 84 and can be opened or breached at a selected time.

Still referring to FIG. 2, in some embodiments, analysis end 34 of test chamber 28 comprises a generally light transparent or laser emission transparent window 90. In some embodiments, window 90 can be transparent to the energy emitted by laser 42 and any resultant emission from a probe contained in sample 110. In some embodiments, test chamber 28 can be provided in a selected orientation such that window 90 can be positioned near camera 50 or other appropriate instrument to detect emission from the selected probe. In some embodiments, window 90 can serve as lens 48 and/or optical filter 52. For example, window 90 can permit a particular wavelength to pass or be altered to permit different wavelengths to pass at different times.

In some embodiments, test chamber 28 comprises a closable door or sealing portion 92 to seal or otherwise contain sample 110, which comprises target sample 110_{T}, within sample chamber 70. This arrangement can, at least in part, permit test chamber 28 to be manipulated, such as via motor 24, without losing sample 110 from test chamber 28.

In some embodiments, electrodes may be used to move at least a portion of sample 110 through test chamber 28. In some embodiments, a sample chamber electrode 94 can be provided near sample chamber 70, a lysing chamber electrode 96 can be provided near lysing chamber 72, a PCR chamber electrode 98 can be provided near PCR chamber 76, and/or a ligation chamber electrode 100 can be provided near ligation chamber 80. In some embodiments, electrodes 94, 96, 98, and 100 can provide a selected charge near respective electrodes 94, 96, 98, and 100 to move a charged portion of sample 110 relative to respective electrodes 94, 96, 98, and 100.

In some embodiments, genomic analyzer 20 can be easily transported and used with generally little input from a user. Generally, in some embodiments, once sample 110 is positioned in sample chamber 70, genomic analyzer 20 can generally operate automatically to perform the various procedures and analyses on sample 110. Therefore, in some embodiments, sample 110 can be prepared in sample chamber 70 and at least a portion of sample 110 can be lysed in lysing chamber 72. In some embodiments, a Polymerase Chain Reaction can be performed in PCR chamber 76 and target sample 110₊ can be ligated to a selected probe in ligation chamber 80. Finally, detection and/or identification of a selected target, such as with a probe, can occur in detection chamber 84 with generally little physical input or output from test chamber 28. In some embodiments, test chamber 28 can replace a plurality of systems, such as a sample preparation or a PCR system. Also, in some embodiments, the need for a transportation or handling system, such as a liquid handler, can be eliminated.

With reference to FIG. 3, in some embodiments, sample chamber 70 of test chamber 28 can be initially filled with sample 110. Sample 110 can be injected in the direction of arrow 112 to position a selected volume within sample chamber 70. In some embodiments, sealing portion 92 can be positioned over sample chamber 70 after positioning sample 110 in sample chamber 70.

In some embodiments, sample 110 can be prepared prior to filling sample chamber 70. For example, in some embodiments, sample 110 can be initially cleaned. In addition, sample 110 can also be pre-crushed, partially digested, such as with a selected chemical complex. Furthermore, in some embodiments, sample chamber 70 can comprise a plurality of portions, such as chemical preparations, to assist in preparing sample 110.

Sample 110 can be placed into the sample chamber in any appropriate manner. In some embodiments, sample 110 can be injected, pipetted, and/or poured into sample chamber 70. Regardless, sample 110 can be positioned into sample chamber 70 in a generally gross manner—That is, sample 110 can comprise portions other than target sample 110_{T}.

In some embodiments, test chamber 28 can be driven by motor 24 once sample 110 is positioned in sample chamber 70 and sealing portion 92 is positioned to close sample chamber 70. Test chamber 28 can be rotated to assist in mixing or separating various portions of sample 110. As discussed herein, sample chamber 70 of test chamber 28 can be driven into position near mixer 36. In some embodiments, mixer 36 can produce an ultrasound wave 114, illustrated diagrammatically in FIG. 3, to assist in preparing and/or separating sample 110 in sample chamber 70. For example, a substantially gross sample, such as a portion of beef meat, can be positioned in sample chamber 70. Target sample 110₊ (also referred to as an organism or microorganism of interest) may not be separated from sample 110 even though the sample may be ground, pulverized, or otherwise broken up before being positioned in sample chamber 70. Therefore, mixer 36 can assist in separating target sample 110₊ from the cells of sample 110.

Once a period of agitation or separation has occurred in sample chamber 70 due to mixer 36, in some embodiments, target sample 110₊ can be at least partially separated from the other cells of sample 110 by urging the sample past or through first filter 74. First filter 74 can be any appropriate screen, gel, or membrane that comprises a pore or hole size that is smaller than the size of the non-target portion of sample 110, but larger than the size of target sample 110_{T}. Selecting an appropriate pore size can assist in separating target sample 110₊ in that a smaller target sample 110₊ can pass through first filter 74 while larger non-target portions of sample 110 are prevented from passing therethrough. In some embodiments, the pores in first filter 74 can be any appropriate size, such as less than 10 micrometers or, more particularly, less than 5 micrometers. In some embodiments, the pore size of first filter 74 can be approximately 10 nanometers to about 10 micrometers.

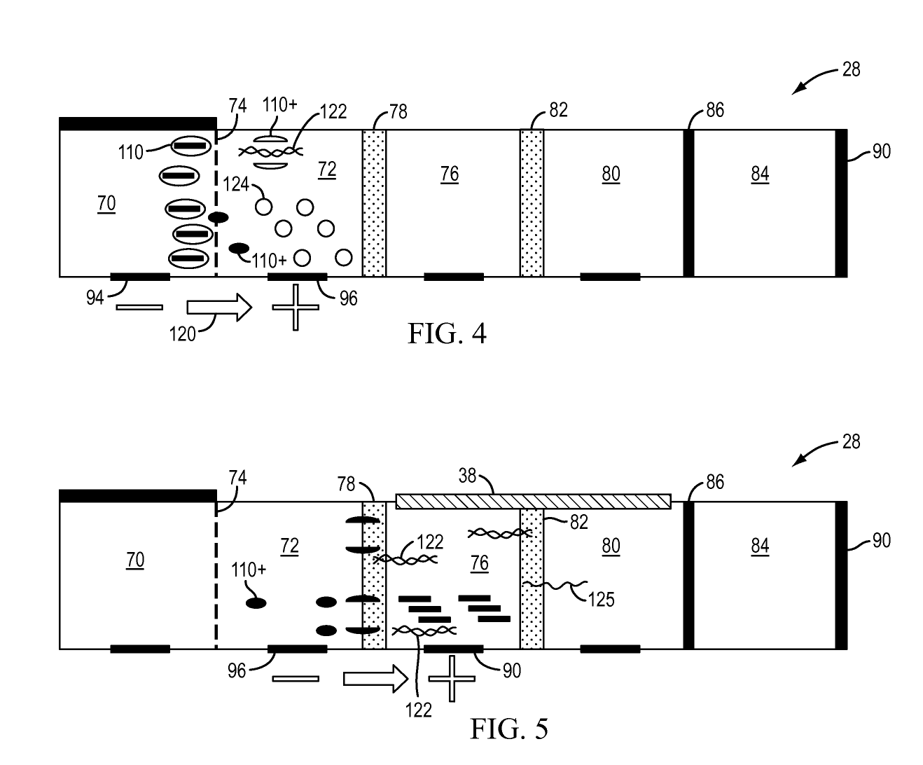

To assist in passing the sample through first filter 74, test chamber 28 can be rotated, in some embodiments, to permit gravity to assist in moving the sample in the direction of arrow 120, as seen in FIG. 4. As sample 110 engages first filter 74, the larger cells of sample 110 are held within sample chamber 70 and target sample 110₊ is permitted to pass through first filter 74 into lysing chamber 72.

In some embodiments, electrophoresis can be used, either alone or in combination with gravity, to assist in moving target sample 110₊ from sample chamber 70 to lysing chamber 72. That is, lysing chamber electrode 96 can be negatively charged while lysing chamber electrode 96 can be positively charged so as to repel and attract, respectively, target sample 110_{T}. As is known in the art, natural or mammalian cells are typically negatively charged and can migrate from sample chamber 70 to lysing chamber 72 due to lysing chamber electrode 96 being positively charged. In some embodiments, a carrier can be provided in sample chamber 70 that includes a selected charge or ion to assist in the migration of target sample 110_{T}. Although all of sample 110 may attempt to move into lysing chamber 72 under electrophoresis, first filter 74 can be used to limit what target sample 110₊ can pass therethrough. In some embodiments, test chamber 28 can be moved to move the large cells of sample 110 away from first filter 74 so as to unclog first filter 74, if needed. In some embodiments, other mechanisms can be used to assist in separating target sample 110₊ from other portions of sample 110.

It should be understood that target sample 110₊ can be organisms of various sizes. For example, target sample 110₊ can be a bacterium or virus. In addition, target sample 110₊ can be a multi-cellular organism that can be separated with first filter 74 or separated in any other appropriate manner from sample 110. Notwithstanding, the organism of interest or target sample 110₊ can be moved into lysing chamber 72 for lysing.

Lysing of target sample 110₊ can occur in any appropriate manner. For example, in some embodiments, a chemical lysing may be used. In some embodiments, various chemical reagents can separate and break apart target sample 110_{T}, including PrepMAN™ Ultra Sample Preparation Reagents from Applied Biosystems of Foster City, Calif. These various chemical reagents can assist in separating a genomic DNA 122 from target sample 110_{T}. In some embodiments, thermocycler 38 can be used to assist in the activity of the various preparation or lysing chemicals.

In some embodiments, mechanical means can be used to lyse target sample 110_{T}. For example, a lysing bead 124 and/or a plurality of lysing beads 124 can be provided in lysing chamber 72. Lysing beads 124 can physically lyse or break apart target sample 110₊ to permit for a release of genomic DNA 122. In some embodiments, test chamber 28 can be moved with motor 24 to assist lysing beads 124 in contacting and lysing target sample 110_{T}.

With reference to FIG. 5, after target sample 110₊ has been lysed in lysing chamber 72, genomic DNA 122 can be moved to PCR chamber 76. As discussed herein, various mechanisms can be used to move genomic DNA 122 from lysing chamber 72 to PCR chamber 76. In some embodiments, test chamber 28 can be moved to permit gravity to assist in moving the material from lysing chamber 72 towards second filter 78. In addition, lysing chamber electrode 96 can be negatively charged and PCR chamber electrode 98 can be positively charged to move target sample 110_{T}. As discussed herein, the natural material of genomic DNA 122 and target sample 110₊ can be negatively charged. Therefore, providing a voltage across second filter 78 can permit a migration of the material towards and through second filter 78. Second filter 78 can permit target sample 110₊ and genomic DNA 122 to pass through second filter 78. In some embodiments, second filter 78 generally permits genomic DNA 122 to pass into PCR chamber 76 while withholding other material of target sample 110_{T}.

In some embodiments, second filter 78 can be any appropriate material, such as a gel matrix. The gel matrix can be similar to any gel matrix that is appropriate for separating various portions, such as genomic portions from other materials. In some embodiments, second filter 78 can permit genomic DNA 122 to pass into PCR chamber 76 while substantially preventing the cell structure of target sample 110₊ from passing therethrough. Hence, second filter 78 can assist in purifying and concentrating a selected portion of sample 110 for analysis in PCR chamber 76.

Furthermore, in some embodiments, second filter 78 can ensure that reactants in lysing chamber 72 can be maintained separate from PCR chamber 76. In some embodiments, this serves, in part, to prevent or otherwise minimize chemical lysing agents in lysing chamber 72 from interfering with any polymerase chain reactions in PCR chamber 76.

In some embodiments, PCR chamber 76 comprises all of the components and reagents necessary to perform amplification, such as PCR. In some embodiments, various components in PCR chamber 76 can be specific to genomic DNA 122. In some embodiments, PCR chamber 76 comprises a polymerase enzyme, forward and reverse primers, and deoxynucleotide triphosphates (dNTPs). These various components can permit amplification of a selected or target genomic DNA. For example, the primers can be provided and designed to hybridize to a specific sequence of genomic DNA 122. In addition, in some embodiments, a plurality of unique primer pairs can be used in PCR chamber 76 to permit multiplexing PCR, thus amplifying a plurality of target regions from a plurality of unique target samples 110₊ present in sample 110. Accordingly, genomic analyzer 20 can be used, in some embodiments, to detect, identify, and/or analyze the presence of more than one selected target sample 110_{T}.

After genomic DNA 122 is passed into PCR chamber 76, thermocycler 38 can be cycled to assist in reacting the various components in PCR chamber 76 to perform PCR of genomic DNA 122. Any number of cycles can be used, such as about 5 to about 100 cycles. In some embodiments, thermocycler 38 can increase, hold, and decrease the temperature of PCR chamber 76 to any appropriate temperature. In some embodiments, it will be understood that a cooling system can also be included in genomic analyzer 20 to cool PCR chamber 76, or any portion of test chamber 28, according to a selected cycle. Nevertheless, PCR chamber 76 can be cycled to a temperature of at least about 95° C. and cooled to about 55° C.

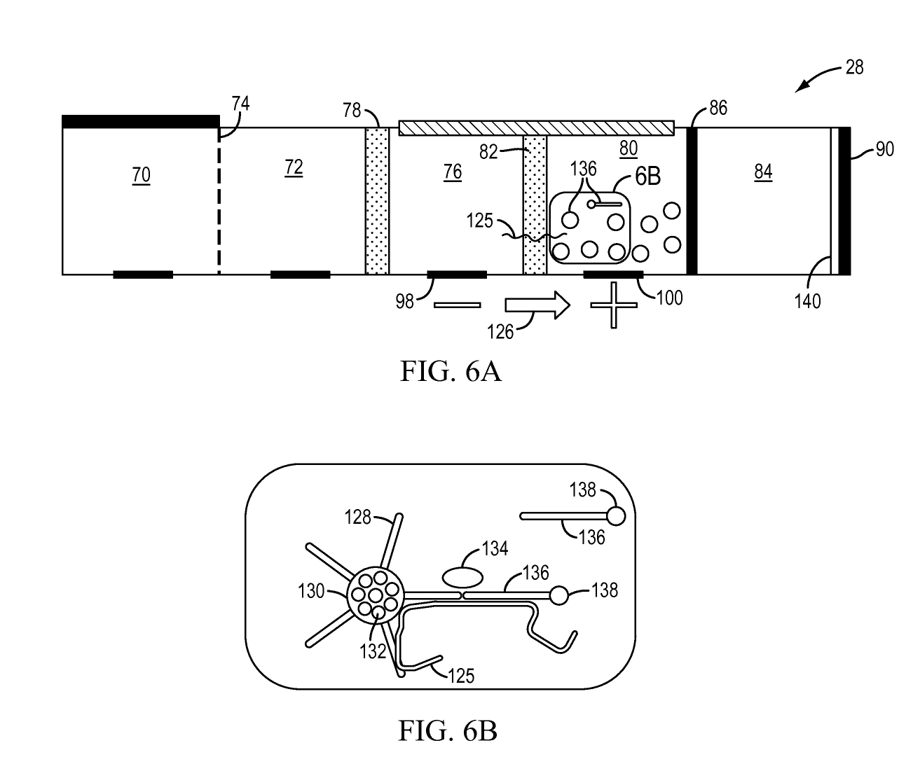

Once amplification has occurred, the amplified sequence of genomic DNA 122 forms an amplicon 125. Amplicon 125 is formed when target sample 110₊ is present in sample 110. If target sample 110₊ is not present in sample 110, then amplicon 125 may not be formed because the various specific primers are not used. As discussed herein, this can indicate whether target sample 110₊ is present in sample 110. Notwithstanding, the material from PCR chamber 76, which comprises amplicon 125, can be passed into ligation chamber 80. With reference to FIG. 6A, in some embodiments, amplicon 125 can be moved in the direction of arrow 126 into ligation chamber 80.

In some embodiments, amplicon 125 can be passed through third filter 82 into ligation chamber 80 via gravity. In some embodiments, amplicon 125 can be urged towards ligation chamber 80 via chamber electrode 100. That is, electrophoresis can assist in moving amplicon 125 from PCR chamber 76 into ligation chamber 80. It should also be understood that other genetic portions, in addition to amplicon 125, can be moved into ligation chamber 80. In some embodiments, third filter 82 can generally separate the components of PCR chamber 76 from the components of ligation chamber 80. Therefore, the portions generally present in ligation chamber 80 can, in some embodiments, comprise amplicon 125 and various components useful for ligation.

In some embodiments, a first ligation probe 128 comprising a code bead 130 that comprises a selected fluorescent dye 132, can be disposed in ligation chamber 80. In some embodiments, one or more dyes 132 can also be placed in ligation chamber 80. In some embodiments, dyes 132 can be activated with laser light energy from laser 42 to fluoresce as described herein. In some embodiments, ligation probe 128 can be ligated to the selected portion of amplicon 125 with a lygase 134. Lygase 134 can also be present in ligation chamber 80.

In addition to first ligation probe 128, in some embodiments, a second ligation probe 136 can be disposed in ligation chamber 80. Second ligation probe 136 can be interconnected with and/or include a biotin portion 138. In some embodiments, first ligation probe 128 and second ligation probe 136 can be sequentially ligated to amplicon 125. Accordingly, a biotin portion 138 can be interconnected with code bead 130 when amplicon 125 is present in sample 110. If biotin portion 138 is interconnected with code bead 130, code bead 130 becomes a biotinylated bead 130a. Additionally, in some embodiments, thermocycler 38 can be used to establish a selected temperature in ligation chamber 80 to permit the ligation to occur.

In some embodiments, an oligonucleotide ligation assay can be used to bind ligation probes 128, 136. Generally, the sequences of ligation probes 128, 136 can be different from the sequences of the PCR primers. Therefore, the PCR primers need not be removed for the ligation reaction to occur properly. This can assist in the ligation being specific to target sample 110₊ and can reduce or eliminate a false positive analysis.

In some embodiments, second ligation probe 136, including biotin portion 138, can be connected to first ligation probe 128 when amplicon 125 of target sample 110₊ is present. In some embodiments, the number of biotinylated beads 130a or the number of biotin associated with a particular code bead 130 can be increased by cycling the ligation process. In some embodiments, thermocycler 38 can be used to denature second ligation probe 136 and first ligation probe 128 and permit rehybridization of probes 128, 136. For example, the temperature in ligation chamber 80 can be increased to a temperature sufficient to permit denaturing of second ligation probe 136 and first ligation probe 128. The temperature can then be varied to the hybridization temperature and permit additional hybridization to occur. After a selected number of hybridization cycles, additional processes can then proceed.

In some embodiments, it should be understood that second ligation probe 136 can be ligated to first ligation probe 128 if amplicon 125 of target sample 110₊ is present. As discussed herein, the various primers present in PCR chamber 76 include those that may react with target sample 110_{T}'s genomic DNA 122. Therefore, in some embodiments, the primers present in PCR chamber 76 do not amplify undesired genomic regions. PCR chamber 76 can generally amplify target sample 110_{T}'s genomic DNA 122. Amplicon 125 generally includes the genomic DNA of target sample 110_{T}. Therefore, test chamber 28 can be designed for a selected genomic sequence, such as one of target sample 110_{T}. Nevertheless, as discussed herein, PCR chamber 76 can comprise a plurality of primers associated with a plurality of organisms such that a multiplexing of the PCR can occur. The multiplexing can permit a plurality of different genomic regions of a plurality of microorganisms to be amplified simultaneously to form a plurality of amplicon 125.

Similarly, in some embodiments, first ligation probe 128 can be specific to target sample 110₊ and/or the genomic portion of target sample 110_{T}. Similarly, second ligation probe 136 can also be specific to target sample 110₊ and/or the genomic portion of target sample 110_{T}. If amplicon 125 of target sample 110₊ is present, first ligation probe 128 can be connected to second ligation probe 136 to form biotinylated bead 130a. This can also be substantially multiplexed due to the specificity of first ligation probe 128 and second ligation probe 136. In some embodiments, a plurality of different specific probes can be provided in ligation chamber 80 so that a plurality of unique microorganisms can be detected and/or identified simultaneously. In some embodiments, the interconnection of first ligation probe 128 and second ligation probe 136 can occur when amplicon 125 is a specific and/or selected target from target sample 110_{T}.

Once the ligation process or steps have occurred, in some embodiments, the biotin can be permitted to interact with a layer or coating of streptavidin 140 (FIG. 6(a)) on window 90. If code beads 130 are formed into biotinylated beads 130a, biotinylated beads 130a can interconnect or react with streptavidin coating 140. The reaction of streptavidin with the biotin can be according to any appropriate mechanism such as that disclosed in U.S. Patent Application Publication US 2003/0165935, published Sep. 4, 2003 and International Publication No. WO 03/045310, published Jun. 5, 2003, each of which is incorporated herein by reference. Generally, biotin portion 138 can interconnect or interact with the streptavidin of streptavidin coating 140 and hold biotin portion 138 to streptavidin coating 140. Therefore, biotinylated beads 130a, which have been biotinylated with second ligation probe 136, can be held near streptavidin coating 140 on window 90.

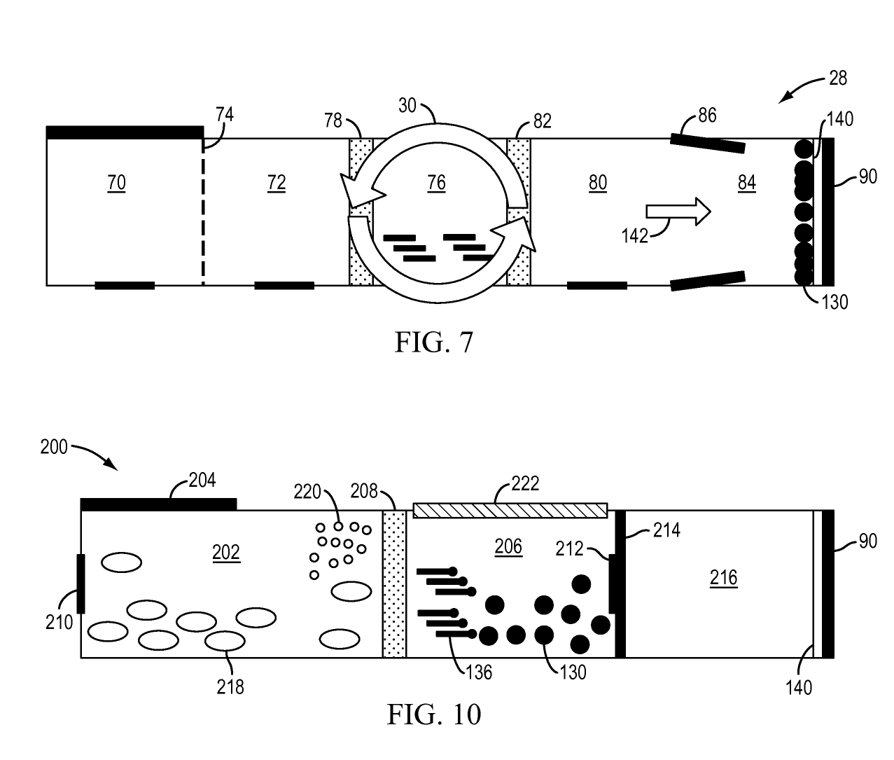

With reference to FIG. 7, as discussed herein, test chamber 28 can be moved in the direction of arrow 30 with motor 24 at any appropriate velocity. This movement of test chamber 28 can generally permit, force, or urge code beads 130, whether biotinylated or not, toward window 90 and/or streptavidin coating 140. In some embodiments, door 86 separating ligation chamber 80 and detection chamber 84 can be opened or broken in any appropriate manner. In some embodiments, door 86 can be opened by centrifugal force of test chamber 28. In some embodiments, door 86 can be actively opened prior to centrifugation. The centrifugal force, generally in the direction of arrow 142, can force code beads 130 toward window 90. It will be understood that code beads 130 are shown diagrammatically and are generally microscopic. In some embodiments, window 90 can be sized to permit at least most of the beads that are originally present in ligation chamber 80 to reach and interact with streptavidin coating 140 on window 90. This permits interaction of at least a majority of the possible biotinylated beads 130a with streptavidin coating 140.

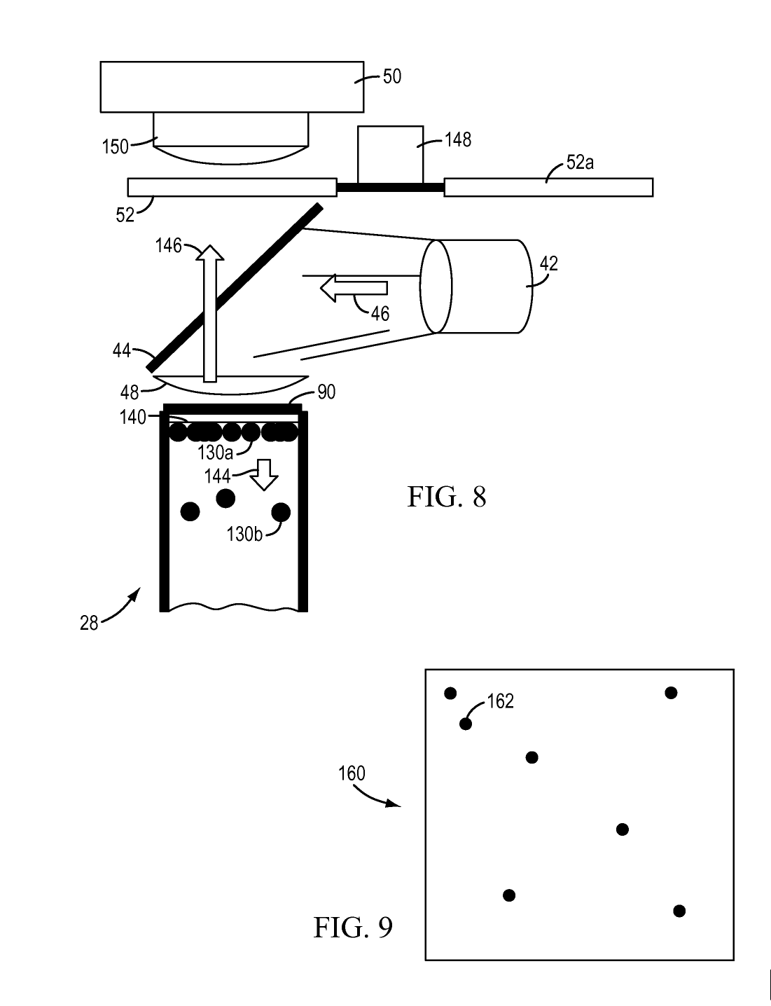

With reference to FIG. 8, after the centrifugation step, test chamber 28 can be positioned substantially in line with the force of gravity in the direction of arrow 144. In this regard, test chamber 28 can be positioned such that window 90 is generally adjacent camera 50. In this position, code beads 130 can be pulled in the general direction of arrow 144 under force of gravity, provided the force of gravity is greater than the contact force between code beads 130 and streptavidin coating 140. That is, unbiotinylated beads 130b can be moved in the direction of arrow 142 away from window 90. This occurs, at least in part, because biotin portion 138 formed on second ligation probe 136 has adhered biotinylated beads 130a to streptavidin coating 140. As discussed herein, the interaction of the biotin and the streptavidin permits fixation of biotinylated beads 130a to window 90.

After a selected period of time sufficient for unbiotinylated beads 130b to move a selected distance away from window 90, laser 42 can be activated to produce laser beam 46. In some embodiments, laser beam 46 can be reflected by dichroic mirror 44 to reflect into window 90 to illuminate or irradiate biotinylated beads 130a. In some embodiments, laser beam 46 can be selected to substantially irradiate biotinylated beads 130a with different wavelengths of energy. In some embodiments, biotinylated beads 130a comprise selected dyes 132 that produce an output emission (i.e. florescence) after being irradiated with laser beam 46. In some embodiments, this output emission from biotinylated beads 130a, generally indicated at 146, can travel and pass through dichroic mirror 44. In some embodiments, dichroic mirror 44 can be selected such that it reflects the wavelength of laser beam 46, but permits output emission to pass therethrough.

In some embodiments, output emission 146 travels toward camera 50 through optical filter 52. Optical filter 52 can permit a selected wavelength of output emission 146 to reach camera 50. In some embodiments, a second optical filter 52a or any appropriate number of optical filters 52 can be provided and a motor 148 can be provided to move a selected one of optical filters 52, 52a into the path of a lens 150 of camera 50. Therefore, output emission 146 that reaches camera 50 can be changed and selected using one or more optical filters.

In some embodiments, as camera 50 receives output emission 146 or a portion thereof that has been filtered by optical filter 52, camera 50 can output a signal to a processor (not shown). With reference to FIG. 9, an exemplary image 160 of output emission 146 is illustrated. In some embodiments, image 160 can comprise a plurality of points or pixel assemblies 162. It should be understood that exemplary image 160 can vary depending upon which filters 52, 52a are passed between lens 150 of camera 50 and window 90 and the wavelengths of energy present in output emission 146. Because probes can be specific and each of the beads comprises a unique fluorescing wavelength, the different wavelengths of energy can be coordinated with a selected probe, and thus, a selected target amplicon 125 of target sample 110_{T}.

In some embodiments, image 160, produced by camera 50, can be processed with the processor to determine the number of pixel assemblies 162 that are present. In some embodiments, the processor can determine the presence of a plurality of pixel assemblies 162 and produce a discrete number that is a determination of the number of pixel assemblies 162. It will be understood that, in some embodiments, pixel assemblies 162 can comprise only one pixel that is captured after being produced by output emission 146 from biotinylated beads 130a.

In some embodiments, from this number of pixel assemblies, the processor can produce a discrete number that represents the number of biotinylated beads 130a. As discussed herein, image 160 may be selectively limited to the wavelength of the output emission that can pass through selected optical filter 52. It will be understood that, in some embodiments, the processor or camera 50 may be used to determine a wavelength detected by pixel assembly 162, thus eliminating the need for optical filters 52, 52a. Therefore, a minimal amount of biotinylated beads 130a, including selected dye 132, can be used to determine the presence of target sample 110_{T}. Various digital detection techniques, generally referred to as digital assays, are known such as those taught in U.S. patent application Ser. No. 10/302,688 (U.S. Patent Application Publication No. 2003/0165935), filed Nov. 21, 2002, entitled, “Digital assay” which is incorporated herein by reference. Thus, a user using genomic analyzer 20 can determine the presence of target sample 110₊ when only a small number of microorganisms are present in sample 110.

In some embodiments, the plurality of code beads 130, which each comprises a different and selected dye 132, can assist in detecting a plurality of unique target samples 110_{T}. For example, a first microorganism can generally interact with a code bead 130 that includes a red dye while a second microorganism can generally interact with a code bead 130 that includes a blue dye. This generally specific interaction can be limited through the tailoring of first ligation probe 128 and second ligation probe 136. Therefore, in some embodiments, the biotinylation of the plurality of code beads 130, wherein each comprises a different selection of dyes 132, can permit a selected microorganism to be interconnected with a selected one of code beads 130. Optical filter 52, 52a can permit camera 50 to gather a plurality of different images by permitting a selected wavelength or bandwidth of energy through to camera 50 that differs depending upon the selected optical filter 52, 52a. The processor can digitally and discretely count the number of pixel assemblies 162 in image 160 thereby permitting determination of whether a selected microorganism is present.

In some embodiments, digital image 160 permits a generally small number of target sample 110₊ to be present in sample 110, but still be detected. For example, it has been discovered that less than about 5,000 individuals of target sample 110₊ can be present and still be detected in image 160, even without amplification. In fact, even fewer numbers of target samples 110₊ are needed if amplification techniques are used. For example, employing ten cycles of PCR produces about a 1024 amplification, therefore generally less than 100, and possibly less than 10, of target sample 110₊ can be present in sample 110, yet permit detection and identification of target sample 110_{T}. Additionally, specificity of code beads 130, including the plurality of dyes 132, can also assist in detection of a small number of target sample 110_{T}. Genomic analyzer 20 can use portions, such as the plurality of filters 52, 52a to filter the plurality of the colors of code beads 130 such that they can be individually imaged in image 160. Thus, genomic analyzer 20, in some embodiments, can provide a specific detection and/or identification of a selected microorganism while only a small number of the microorganisms are present in sample 110.

Genomic analyzer 20, in some embodiments, can be used for a plurality of analyses. The PCR portion of test chamber 28 can provide an amplification of a selected target, such as target sample 110_{T}. The amplification can increase the number of targets by forming amplicon 125 that can interact with first and second probes 128, 136. Therefore, initial sample 110 comprises a limited number of target sample 110_{T}, yet target sample 110₊ can still be detected and/or identified.

In some embodiments, the processing time can be decreased and efficiency of genomic analyzer 20 can be increased by not using the separate lysing chamber 72 and PCR chamber 76. It is generally understood that cycles of PCR can be performed to increase or amplify the number of a selected portion of the sample, such as genomic DNA 122. Therefore, if an ample or a selected number of the organisms of interest are present in gross sample 110, the PCR cycles may be eliminated yet an appropriate amount of target sample 110₊ is still present.

With reference to FIG. 10, in some embodiments, a test chamber or test chamber 200 can comprise a sample and/or lysing chamber 202 that can be sealed with a cap or sealing portion 204. In some embodiments, test chamber 200 can also comprise a ligation chamber 206 that can be separated from lysing chamber 202 with a filter 208. In some embodiments, test chamber 200 can comprise a first electrode 210 positioned near lysing chamber 202 and a second electrode 212 positioned near ligation chamber 206. A breakable or moveable door 214 can separate ligation chamber 206 from a detection chamber 216. Detection chamber 216 can be terminated with window 90 that can be covered with streptavidin coating 140, as discussed herein. In some embodiments, test chamber 200 can be provided without a sample chamber that is physically separated from lysing chamber 202 or a PCR chamber in which PCR could occur.

In some embodiments, test chamber 200 can be used in a manner generally similar to test chamber 28. That is, test chamber 200 can be positioned within genomic analyzer 20 and interconnected with chamber grasping system 26 to be moved with motor 24. Generally, test chamber 200 can be operated within genomic analyzer 20 to perform a selected analysis that can be performed quicker than the analysis performed with test chamber 28. In part, this can be due to the elimination of PCR chamber 76 and the lack of the step of separating target sample 110₊ from gross sample 110. Therefore, a sample comprising a plurality of organisms 218 can be positioned within lysing chamber 202 that is sealed with the cap.

Because sample 218 generally includes an appropriate amount of the target sample, sample 218 can also be referred to as the target sample. Also, in some embodiments, lysing chamber 202 can be the first chamber into which sample 218 is positioned. In lysing chamber 202, sample 218 can be lysed, as discussed herein. For example, a lysing bead 220 can be provided in lysing chamber 202 and test chamber 200 can be moved to permit substantial mechanical lysing of the sample. In some embodiments, various chemical reagents can be provided to lyse sample 218. Regardless, as discussed herein, substantially lysing sample 218 permits for freeing of the genomic DNA, RNA, or other sequence including portions of sample 218.

Once the genomic DNA has been removed from sample 218, the genomic DNA can be separated from the remaining cellular material and moved into ligation chamber 206 through filter 208. As discussed herein, filter 208 can be any appropriate separating mechanism, such as a gel matrix. In some embodiments, the genomic DNA can be moved into ligation chamber 206 using any appropriate mechanism. For example, first electrode 210 can be negatively charged and second electrode 212 can be positively charged such that the generally naturally negatively charged material can be urged toward positively charged electrode 212. Filter 208 can permit the genomic DNA to pass through filter 208 to ligation chamber 206.

It will be understood that lysing chamber 202 and ligation chamber 206 can also be a single chamber, in some embodiments. Therefore, depending on the sample positioned in test chamber 200, lysing chamber 202 and ligation chamber 206 can be a single chamber. This can permit the sample to be efficiently positioned in test chamber 200, lysed and then generally immediately ligated without being separated from the other cellular material.

Once the genomic DNA is in ligation chamber 206, ligation of the genomic DNA may occur, as discussed herein. Generally, first ligation probe 128, interconnected with code bead 130, can be hybridized with a ligase to the genomic DNA segment. Second ligation probe 136, including biotin portion 138, can also be ligated to the genomic DNA portion. As discussed, this permits code bead 130 to be interconnected with biotin portion 138 by interconnection of first ligation probe 128 and second ligation probe 136. The hybridization can be performed at a selected temperature, such as about 50° to 60° C. or about 55° C. The hybridization temperature can be created with a heating or temperature control mechanism 222. In some embodiments, temperature control mechanism 222 can comprise a resistive strip that is positioned relative to ligation chamber 206 to heat ligation chamber 206 to a selected temperature. It will be understood that thermocycling may not be necessary and temperature control mechanism 222 can be provided relative to ligation chamber 206 to maintain a hybridization temperature in ligation chamber 206.

In some embodiments, the hybridization of first ligation probe 128 and second ligation probe 136 can permit the formation of a plurality of the biotinylated beads 130a. In some embodiments, test chamber 200 can then be centrifuged following hybridization of the genomic DNA and formation of biotinylated beads 130a. As discussed herein, door or wall 214 can be opened actively or due to the force of the centrifugation. Opening door 214 permits biotinylated beads 130a or all the beads to move towards window 90 through detection chamber 216.

As discussed herein, generally digital assay analysis can be used to perform a detection and/or identification of an organism of interest due to a wavelength or selected brightness of dye 132, or other detectable portion, in code beads 130. Therefore, if the genomic portion is present, code bead 130 becomes biotinylated with biotin portion 138, due to the ligation and interconnection of first ligation probe 128 with second ligation probe 136. This permits a generally small number of selected genomic portions to form a positive or negative determination of a presence of an organism of interest. Therefore, in some embodiments, test chamber 200 can be used when the organism of interest is positioned in lysing chamber 202 and is generally not necessary to be separated from a host organism or structure. However, as discussed herein, in some embodiments, the separation of lysing chamber 202 from ligation chamber 206 can be eliminated for various applications. This will permit lysing of cellular structure 218 to achieve access to the genomic DNA that can be ligated with probes 128, 136 in a single area.

It will be understood that genomic analyzer 20 and test chambers 28, 200 can be any appropriate size. For example, the size of test chamber 28, 200 can be chosen depending on the size or volume of the sample to be tested, the required time for completing the analysis, or the type of analysis to be performed. Generally, in some embodiments, test chambers 28, 200 can comprise a volume of about 1 ml to about 500 ml. However, any size can be chosen for various applications, efficiencies, or other considerations. Moreover, sample 110, 218 can be provided either alone or in a selected carrier or solution. The carrier may assist in the preparation and analysis, such as separating the various portions.

1.-18. (canceled)

19. A processing and analysis apparatus comprising:
a test chamber having at least a sample chamber and a detection chamber defined therein, said sample chamber being sized to receive a sample therein;
a first filter being disposed between and separating said sample chamber and said detection chamber;
a drive mechanism being operably coupled with said test chamber, said drive mechanism selectively rotatably driving said test chamber; and
an analysis system being positioned adjacent said test chamber, said analysis system being operably to perform a selected analysis or procedure.

20. The apparatus of claim 19 wherein said analysis system comprises:
a processing unit; and
a power source operably coupled to said processing unit for providing power to said processing unit.

21. The apparatus of claim 20 wherein said analysis system comprises:
an imaging device operably coupled with said power source, said imaging device positioned to receive an output emission emanating from said test chamber.

22. The apparatus of claim 21, further comprising:
an output window disposed at an end of said detection chamber, said output window permitting at least a portion of said output emission to pass therethrough.

23. The apparatus of claim 22, further comprising:
a first probe;
a second probe connected to a biotin portion;
said first probe and said second probe being selectively ligated to form a biotinylated bead.

24. The apparatus of claim 23 wherein said biotinylated bead is affixed to said window.

25. The apparatus of claim 21 wherein said imaging device is a digital imaging device.

26. The apparatus of claim 21 wherein said analysis system comprises:
an optical filter disposed between said test chamber and said imaging device, said optical filter permitting only a predetermined wavelength of said output emission to pass therethrough.

27. The apparatus of claim 20 wherein said analysis system comprises:
a laser operably coupled with said power source, said laser selectively outputting a laser beam.

28. The apparatus of claim 27 wherein said analysis system comprises:
a dichroic mirror disposed between said laser and said test chamber, said dichroic mirror directing said laser beam along a predetermine path.

29. The apparatus of claim 20 wherein said analysis system comprises:
an analysis output system operably coupled with said processing unit, said analysis output system selectively outputting results from said selected analysis or procedure.

30. The apparatus of claim 19, further comprising:
a lysing chamber being positioned between said sample chamber and said detection chamber such that said first filter is disposed between said sample chamber and said lysing chamber.

31. The apparatus of claim 30, further comprising:
an amplification chamber being positioned adjacent said lysing chamber; and
a second filter being disposed between said lysing chamber and said amplification chamber.

32. The apparatus of claim 31 wherein at least one of said first filter and said second filter is a gel matrix.

33. The apparatus of claim 31 wherein at least one of said first filter and said second filter define a pore size of about one micrometer to about one millimeter.

34. The apparatus of claim 31, further comprising:
a ligation chamber being positioned between said amplification chamber and said detection chamber;
a third filter being disposed between said amplification chamber and said ligation chamber; and
a member being disposed between said ligation chamber and said detection chamber, said member being selectively opened.

35. The apparatus of claim 34, further comprising:
a probe interconnected to a bead including a bandwidth emitting substance, said probe being disposed in said ligation chamber.

36. The apparatus of claim 19, further comprising:
a mixer operably positioned relative to said test chamber so mix said sample in said test chamber.

37. The apparatus of claim 36 wherein said mixer is an ultrasonic mixer.

38. The apparatus of claim 19, further comprising:
a bead disposed in said test chamber;
a probe configured to associated with a portion of said sample, said probe being coupled with said bead; and
a detectable substance operable to output an emission.

39.-43. (canceled)
